SEQUENCE LISTING (24 sequences)

SEQ ID NO: 1 - DNA, 1995 bp, Homo sapiens
  prim_transcript (1)..(1995)
gatgctgccttcccacataaatgagatttttttctgccaggcaacatggttttaccctcatattcaaaagtaagtagctggagcgctggtctttgccagggaaggagtgatccagaagctgcctggcagcattttgtggggctggtcagggaatggggtgtaaatgacaacagatattaagggctcttgtgagtagagcaaggagttgggtacagaatattcttcagctggtctagcagaaatggaatctgcttcctggtttcagctctgcaggcttggtatgtaggatgtctttaagctttatggctgatgccctaaagttctgtgtgtaaggatgctctaaagtgtgaagtacacagctgctgggctgggcaactatagtgttttgggagataaacagggcaagtggcttgtcttaggtcatggtgactggaatgattttcagtactagggcaatcattctgacttaattccaggggtagggtgatgggagttgaggaacctcagtccatccctggctgctgtggactaagcactgactttgacaagctgagactgctaagtctttgtcctgtcctgcccggctgggtagtggggagtaagaagctgaaagggaggtgggactttccacgatagtggcctcctggagcttccactcttctttccctacaggctcatagttcctacacagctactggcttctctgttttgaggcagtttccttcttgggggtttccttgataaagttatgggcttgggtgcccattgtcccccatgccactgagcttgttctagagttcgaggaccatagaaggggcctccaaagattccttctgggatctttccccattatcttttcatcctaccagtcagagggagggtcattattggatatctactgtttactcacgtattggatggaggtggtgcccaccctcttggcagagacaaagattccagccactgatgtcgctgatgccagcctgaatgaatgttccagtaccgaaaggaaacaagacgtagtgttgctgttcgtgaccttgtcccacacacagccacctctgtttcacctgccttatgtccagaaacccttaatctctaatgtggagcagctgatcctggggatcccgggccagaatcgccgggagataggccatggccaggatatctttccagcagagaagctctgccatctgcaggatcgcaaggtgaaccttcacagagctgcctggggcgagtgtattgttgcacccaagactctcagcttctcttactgtcaggggacctgcccggccctcaacagtgagctccgtcattccagctttgagtgctataaggtaagacatggagcctcgttctttctcttctggggtcatattgggatagcactaagtgctcaactctctaggcctggctccttttgagtcaaggaagccattgaagttggtaattatgtaatctagcactgatgcagtgtgtagcatcttccccgccctgtgaccttatcccttatctttattcataagaaacatcagcttcctaaagattgttctgaaacagccctgatccagcagcttctccccaggccctccttctcccttcccatgtatccctgacaagtctactgatgcccttagatatgaggctgtggctatgaggcactcaccattctgccatttgtttctgcagagggcagtacctacctgtccctggctcttccagacctgccgtcccaccatggtcagactcttctccctgatggtccaggatgacgaacacaagatgagtgtgcactatgtgaacacttccttggtggagaagtgtggctgctcttgagataccccaaagcctcctactggcctcagggccacctaagtctcaggactttagtagggggtgggattacttttcatagcaagtagagctctttgaagggaggtgggatttggtttgtttctcaaagcacagcaagaaggttggcattatggcagtaac

SEQ ID NO: 2 - DNA, 639 bp, Homo sapiens
  CDS (1)..(639): main CDS, encodes NSG3 full-length protein (212 aa)
atggttttaccctcatattcaaaatcagagggagggtcattattggatatctactgtttactcacgtattggatggaggtggtgcccaccctcttggcagagacaaagattccagccactgatgtcgctgatgccagcctgaatgaatgttccagtaccgaaaggaaacaagacgtagtgttgctgttcgtgaccttgtcccacacacagccacctctgtttcacctgccttatgtccagaaacccttaatctctaatgtggagcagctgatcctggggatcccgggccagaatcgccgggagataggccatggccaggatatctttccagcagagaagctctgccatctgcaggatcgcaaggtgaaccttcacagagctgcctggggcgagtgtattgttgcacccaagactctcagcttctcttactgtcaggggacctgcccggccctcaacagtgagctccgtcattccagctttgagtgctataagagggcagtacctacctgtccctggctcttccagacctgccgtcccaccatggtcagactcttctccctgatggtccaggatgacgaacacaagatgagtgtgcactatgtgaacacttccttggtggagaagtgtggctgctcttga

SEQ ID NO: 3 - protein, 212 aa, Homo sapiens
MVLPSYSKSEGGSLLDIYCLLTYWMEVVPTLLAETKIPATDVADASLNECSSTERKQDVVLLFVTLSHTQPPLFHLPYVQKPLISNVEQLILGIPGQNRREIGHGQDIFPAEKLCHLQDRKVNLHRAAWGECIVAPKTLSFSYCQGTCPALNSELRHSSFECYKRAVPTCPWLFQTCRPTMVRLFSLMVQDDEHKMSVHYVNTSLVEKCGCS

SEQ ID NO: 4 - DNA, 585 bp, Homo sapiens
  CDS (1)..(585): alternative CDS, encodes a NSG3 splice variant (194 aa)
atggttttaccctcatattcaaaagtggtgcccaccctcttggcagagacaaagattccagccactgatgtcgctgatgccagcctgaatgaatgttccagtaccgaaaggaaacaagacgtagtgttgctgttcgtgaccttgtcccacacacagccacctctgtttcacctgccttatgtccagaaacccttaatctctaatgtggagcagctgatcctggggatcccgggccagaatcgccgggagataggccatggccaggatatctttccagcagagaagctctgccatctgcaggatcgcaaggtgaaccttcacagagctgcctggggcgagtgtattgttgcacccaagactctcagcttctcttactgtcaggggacctgcccggccctcaacagtgagctccgtcattccagctttgagtgctataagagggcagtacctacctgtccctggctcttccagacctgccgtcccaccatggtcagactcttctccctgatggtccaggatgacgaacacaagatgagtgtgcactatgtgaacacttccttggtggagaagtgtggctgctcttga

SEQ ID NO: 5 - protein, 194 aa, Homo sapiens
MVLPSYSKVVPTLLAETKIPATDVADASLNECSSTERKQDVVLLFVTLSHTQPPLFHLPYVQKPLISNVEQLILGIPGQNRREIGHGQDIFPAEKLCHLQDRKVNLHRAAWGECIVAPKTLSFSYCQGTCPALNSELRHSSFECYKRAVPTCPWLFQTCRPTMVRLFSLMVQDDEHKMSVHYVNTSLVEKCGCS

SEQ ID NO: 6 - DNA, 423 bp, Homo sapiens
  CDS (1)..(423): alternative CDS, encodes a NSG3 splice variant (140 aa)
atggttttaccctcatattcaaaaaaacccttaatctctaatgtggagcagctgatcctggggatcccgggccagaatcgccgggagataggccatggccaggatatctttccagcagagaagctctgccatctgcaggatcgcaaggtgaaccttcacagagctgcctggggcgagtgtattgttgcacccaagactctcagcttctcttactgtcaggggacctgcccggccctcaacagtgagctccgtcattccagctttgagtgctataagagggcagtacctacctgtccctggctcttccagacctgccgtcccaccatggtcagactcttctccctgatggtccaggatgacgaacacaagatgagtgtgcactatgtgaacacttccttggtggagaagtgtggctgctcttga

SEQ ID NO: 7 - protein, 140 aa, Homo sapiens
MVLPSYSKKPLISNVEQLILGIPGQNRREIGHGQDIFPAEKLCHLQDRKVNLHRAAWGECIVAPKTLSFSYCQGTCPALNSELRHSSFECYKRAVPTCPWLFQTCRPTMVRLFSLMVQDDEHKMSVHYVNTSLVEKCGCS

SEQ ID NO: 8 - DNA, 540 bp, Homo sapiens
  CDS (1)..(540): encodes NSG3 pro-protein (179 aa)
gagacaaagattccagccactgatgtcgctgatgccagcctgaatgaatgttccagtaccgaaaggaaacaagacgtagtgttgctgttcgtgaccttgtcccacacacagccacctctgtttcacctgccttatgtccagaaacccttaatctctaatgtggagcagctgatcctggggatcccgggccagaatcgccgggagataggccatggccaggatatctttccagcagagaagctctgccatctgcaggatcgcaaggtgaaccttcacagagctgcctggggcgagtgtattgttgcacccaagactctcagcttctcttactgtcaggggacctgcccggccctcaacagtgagctccgtcattccagctttgagtgctataagagggcagtacctacctgtccctggctcttccagacctgccgtcccaccatggtcagactcttctccctgatggtccaggatgacgaacacaagatgagtgtgcactatgtgaacacttccttggtggagaagtgtggctgctcttga

SEQ ID NO: 9 - protein, 179 aa, Homo sapiens
ETKIPATDVADASLNECSSTERKQDVVLLFVTLSHTQPPLFHLPYVQKPLISNVEQLILGIPGQNRREIGHGQDIFPAEKLCHLQDRKVNLHRAAWGECIVAPKTLSFSYCQGTCPALNSELRHSSFECYKRAVPTCPWLFQTCRPTMVRLFSLMVQDDEHKMSVHYVNTSLVEKCGCS

SEQ ID NO: 10 - DNA, 339 bp, Homo sapiens
  CDS (1)..(339): encodes NSG3 mature protein (112 aa)
gagataggccatggccaggatatctttccagcagagaagctctgccatctgcaggatcgcaaggtgaaccttcacagagctgcctggggcgagtgtattgttgcacccaagactctcagcttctcttactgtcaggggacctgcccggccctcaacagtgagctccgtcattccagctttgagtgctataagagggcagtacctacctgtccctggctcttccagacctgccgtcccaccatggtcagactcttctccctgatggtccaggatgacgaacacaagatgagtgtgcactatgtgaacacttccttggtggagaagtgtggctgctcttga

SEQ ID NO: 11 - protein, 112 aa, Homo sapiens
EIGHGQDIFPAEKLCHLQDRKVNLHRAAWGECIVAPKTLSFSYCQGTCPALNSELRHSSFECYKRAVPTCPWLFQTCRPTMVRLFSLMVQDDEHKMSVHYVNTSLVEKCGCS

SEQ ID NO: 12 - DNA, 291 bp, Homo sapiens
  CDS (1)..(291): encodes a core fragment of NSG3 (aa 15-111 of mature NSG3; 97 aa)
tgccatctgcaggatcgcaaggtgaaccttcacagagctgcctggggcgagtgtattgttgcacccaagactctcagcttctcttactgtcaggggacctgcccggccctcaacagtgagctccgtcattccagctttgagtgctataagagggcagtacctacctgtccctggctcttccagacctgccgtcccaccatggtcagactcttctccctgatggtccaggatgacgaacacaagatgagtgtgcactatgtgaacacttccttggtggagaagtgtggctgc

SEQ ID NO: 13 - protein, 97 aa, Homo sapiens
CHLQDRKVNLHRAAWGECIVAPKTLSFSYCQGTCPALNSELRHSSFECYKRAVPTCPWLFQTCRPTMVRLFSLMVQDDEHKMSVHYVNTSLVEKCGC

SEQ ID NO: 14 - DNA, 300 bp, Homo sapiens
  CDS (1)..(300): encodes an extended core fragment of NSG3 (aa 14-112 of mature NSG3; 99 aa)
ctctgccatctgcaggatcgcaaggtgaaccttcacagagctgcctggggcgagtgtattgttgcacccaagactctcagcttctcttactgtcaggggacctgcccggccctcaacagtgagctccgtcattccagctttgagtgctataagagggcagtacctacctgtccctggctcttccagacctgccgtcccaccatggtcagactcttctccctgatggtccaggatgacgaacacaagatgagtgtgcactatgtgaacacttccttggtggagaagtgtggctgctcttga

SEQ ID NO: 15 - protein, 99 aa, Homo sapiens
LCHLQDRKVNLHRAAWGECIVAPKTLSFSYCQGTCPALNSELRHSSFECYKRAVPTCPWLFQTCRPTMVRLFSLMVQDDEHKMSVHYVNTSLVEKCGCS

SEQ ID NO: 16 - DNA, 555 bp, Homo sapiens
  CDS (1)..(555): encodes a synthetic NSG2 pro-protein (184 aa); stopcodon at position 169-171 of SEQ ID NO 24 changed to CAA/Gln
gaggaaaagattacagctactaatgcgtctgaccccagcctgaaccagtgttttagtatcaaaggcaagcaagacatagtgttgctgttcatgaccttgtccccaacacagccacctctgtttcacctgccttacgtccagaaatgctttatccctactgtggagcagctgactctggggatcccatgccagaatcatggggagatagaccatggccaggatatatttccagcagagaagctctgtcatctgcaggattgcaaggtgaaccttcacagagctgcctgcggtgagtgtattgttgcacccaagacttccagcttcccttactgtcaggggacctgcctgaccctcaacagtgagcttcatcaatccaactttgcactcaaagtttgcactataagaggggagtgcctattgatctgttcctggctctttcagacctgtagtcccaccaaggtcattctcttctccctaacggtccaggatgacgaacgtaagatgagcgttcactgtgtgaacgcatccttgatagagaagtgtggctgctcttga

SEQ ID NO: 17 - protein, 184 aa, Homo sapiens
EEKITATNASDPSLNQCFSIKGKQDIVLLFMTLSPTQPPLFHLPYVQKCFIPTVEQLTLGIPCQNHGEIDHGQDIFPAEKLCHLQDCKVNLHRAACGECIVAPKTSSFPYCQGTCLTLNSELHQSNFALKVCTIRGECLLICSWLFQTCSPTKVILFSLTVQDDERKMSVHCVNASLIEKCGCS

SEQ ID NO: 18 - DNA, 354 bp, Homo sapiens
  CDS (1)..(354): encodes mature form of NSG2 (117 aa)
gagatagaccatggccaggatatatttccagcagagaagctctgtcatctgcaggattgcaaggtgaaccttcacagagctgcctgcggtgagtgtattgttgcacccaagacttccagcttcccttactgtcaggggacctgcctgaccctcaacagtgagcttcatcaatccaactttgcactcaaagtttgcactataagaggggagtgcctattgatctgttcctggctctttcagacctgtagtcccaccaaggtcattctcttctccctaacggtccaggatgacgaacgtaagatgagcgttcactgtgtgaacgcatccttgatagagaagtgtggctgctcttga

SEQ ID NO: 19 - protein, 117 aa, Homo sapiens
EIDHGQDIFPAEKLCHLQDCKVNLHRAACGECIVAPKTSSFPYCQGTCLTLNSELHQSNFALKVCTIRGECLLICSWLFQTCSPTKVILFSLTVQDDERKMSVHCVNASLIEKCGCS

SEQ ID NO: 20 - DNA, 306 bp, Homo sapiens
  CDS (1)..(306): encodes a core fragment of NSG2 (aa 15-116 of mature NSG2; 102 aa)
tgtcatctgcaggattgcaaggtgaaccttcacagagctgcctgcggtgagtgtattgttgcacccaagacttccagcttcccttactgtcaggggacctgcctgaccctcaacagtgagcttcatcaatccaactttgcactcaaagtttgcactataagaggggagtgcctattgatctgttcctggctctttcagacctgtagtcccaccaaggtcattctcttctccctaacggtccaggatgacgaacgtaagatgagcgttcactgtgtgaacgcatccttgatagagaagtgtggctgc

SEQ ID NO: 21 - protein, 102 aa, Homo sapiens
CHLQDCKVNLHRAACGECIVAPKTSSFPYCQGTCLTLNSELHQSNFALKVCTIRGECLLICSWLFQTCSPTKVILFSLTVQDDERKMSVHCVNASLIEKCGC

SEQ ID NO: 22 - DNA, 315 bp, Homo sapiens
  CDS (1)..(315): encodes an extended core fragment of NSG2 (aa 14-117 of mature NSG2; 104 aa)
ctctgtcatctgcaggattgcaaggtgaaccttcacagagctgcctgcggtgagtgtattgttgcacccaagacttccagcttcccttactgtcaggggacctgcctgaccctcaacagtgagcttcatcaatccaactttgcactcaaagtttgcactataagaggggagtgcctattgatctgttcctggctctttcagacctgtagtcccaccaaggtcattctcttctccctaacggtccaggatgacgaacgtaagatgagcgttcactgtgtgaacgcatccttgatagagaagtgtggctgctcttga

SEQ ID NO: 23 - protein, 104 aa, Homo sapiens
LCHLQDCKVNLHRAACGECIVAPKTSSFPYCQGTCLTLNSELHQSNFALKVCTIRGECLLICSWLFQTCSPTKVILFSLTVQDDERKMSVHCVNASLIEKCGCS

SEQ ID NO: 24 - DNA, 654 bp, Homo sapiens
  misc_feature (1)..(654)
atggttttaccctcacatccaaaagcgaaaggaggatcattattggagatctactgtttactaatatattggatggaggtggtgcccaccctcttggcagaggaaaagattacagctactaatgcgtctgaccccagcctgaaccagtgttttagtatcaaaggcaagtgagacatagtgttgctgttcatgaccttgtccccaacacagccacctctgtttcacctgccttacgtccagaaatgctttatccctactgtggagcagctgactctggggatcccatgccagaatcatggggagatagaccatggccaggatatatttccagcagagaagctctgtcatctgcaggattgcaaggtgaaccttcacagagctgcctgcggtgagtgtattgttgcacccaagacttccagcttcccttactgtcaggggacctgcctgaccctcaacagtgagcttcatcaatccaactttgcactcaaagtttgcactataagaggggagtgcctattgatctgttcctggctctttcagacctgtagtcccaccaaggtcattctcttctccctaacggtccaggatgacgaacgtaagatgagcgttcactgtgtgaacgcatccttgatagagaagtgtggctgctcttga

(19) United States
(12) Patent Application Publication
(10) Pub. No.: US 2004/0259779 A1
(43) Pub. Date: Dec. 23, 2004

(54) Novel neurotrophic factors

(76) Inventors: Teit Johansen, Ballerup (DK); Nikolaj Blom, Kobenhavn NV (DK); Claus Hansen, Holbaek (DK)

Correspondence Address: BROWDY AND NEIMARK, P.L.L.C., 624 NINTH STREET, NW, SUITE 300, WASHINGTON, DC, 20001-5303, US

(21) Appl. No.: 10/482,914

(22) PCT Filed: Jul. 8, 2002
(86) PCT No.: PCT/DK02/00475

Related U.S. Application Data
(60) Provisional application No. 60/303,947, filed on Jul. 9, 2001.

(30) Foreign Application Priority Data
Jul. 6, 2001 (DK) PA 2001 01069

(51) Int. Cl. A61K 38/17; C07H 21/04; C07K 14/47

(52) U.S. Cl. 514/12; 530/350; 536/23.5; 435/69.1; 435/320.1; 435/325

(57) ABSTRACT

The present invention relates to novel biologically active, processed forms of the neuronal survival and growth factor protein denominated NSG3, in particular the pro-protein, the mature protein and core fragment of NSG3, as well as variants thereof. Also, the use of the novel polypeptides for treating or preventing a neurodegenerative disease, in particular an excitotoxic disease, a cancer, tissue injury, insufficient bone cartilage growth and maturation or a disease involving muscle in an animal is described.

### TECHNICAL FIELD

The present invention relates to novel polypeptides. More specifically the invention provides novel polypeptides having neurotrophic activity. The novel polypeptides of the invention are believed to belong to a subfamily of the Transforming Growth Factor-β family,

The invention also relates to isolated nucleic acid sequences encoding the novel polypeptides, and to nucleic acid constructs, vectors, and host cells comprising the nucleic acid sequences, as well as methods for producing the novel polypeptides.

### BACKGROUND ART

Neurotrophic factors are proteins which may be isolated from the nervous system or from non-nerve tissues innervated by the nervous system. Neurotrophic factors promote survival and maintain the phenotypic differentiation of nerve cells, thereby preventing degeneration and increasing the functional activity of neuronal tissue.

Different neurotrophic factors affect distinctly different classes of nerve cells, and the neurotrophic factors may thus be classified accordingly. Examples of neurotrophic factor (super)families include the fibroblast growth factor family, the neurotrophin family, and the Transforming Growth Factor-β (TGF-β) family.

This invention relates to polynucleotide and polypeptide molecules which are structurally related to TGF-β family members. The TGF-β family of peptide growth factors have a characteristic fold structure which is held in place by a so-called ‘cysteine knot’ formed from six cysteine residues which are conserved between members of the family despite otherwise low levels of homology. (McDonald N Q et al Cell. 1993 May 7; 73(3): 421-4.)

Members of the TGF-β superfamily have diverse biological activities and play critical roles in the migration, proliferation and differentiation of cells during embryogenesis and in the repair and regeneration of tissues during post fetal life.

The proteins of the TGF-beta family are initially synthesized as large precursor proteins, which subsequently undergo proteolytic cleavage at a cluster of basic residues approximately 110-140 amino acids from the C-terminus. The C-terminal regions, or mature regions, of the proteins are all structurally related and the different family members can be classified into distinct subgroups based on the extent of their homology. Although the homologies within particular subgroups range from 70% to 90% amino acid sequence identity, the homologies between subgroups are significantly lower, generally ranging from only 20% to 50%. The majority of TGF-β family proteins form homo-dimers of approximately 25 kD via an intermolecular disulphide bridge from a seventh conserved cysteine. Glial Cell Line Derived Neurotrophic Factor (GDNF) has recently been shown to dimerise despite the selective reduction and alkylation of this cystine bridge (Hui O. et al. J Protein Chem 1999 July; 18(5): 585-93). The resulting protein was shown to chromatograph as a dimer and displayed similar activity to the native protein. This finding suggests that the TGF-β super-family proteins lacking the seventh cysteine may also form dimers through non-covalent association. The polypeptides of the present invention retain six of the seven cysteine residues conserved in the C-terminal, active domain of TGF-β.

The polypeptide of this invention is closely related to the Glial Cell Derived Neurotrophic Factor (GDNF) sub-family of neurotrophic factors. This family includes GDNF, Neublastin, Persephin or Neurturin. The TGF-β family belongs to a larger, extended super family of peptide signaling molecules that includes bone morphogenic proteins (Wozney, J. M. et al., Science, 242:1528-1534 (1988)), vg1 (Weeks, D. L., and Melton, D. A., Cell, 51:861-867 (1987)), activins (Vale, W. et al., Nature, 321:776-779 (1986)), and inhibins (Mason, A. J. et al., Nature, 318:659-663 (1985)). All of these molecules are thought to play an important role in modulating growth, development and differentiation. Some of the TGF-β proteins have been shown to have a broad spectrum of activity, for example Bone Morphogenic Protein 11 (BMP11) was found to be active in promoting growth and differentiation of neuronal tissue, as well as bone, cartilage and connective tissue (WO99/24057). A review of Bone Morphogenic Proteins (Ducy & Karsenty, Kidney International, 57, 2207-2214 (2000)) notes that, despite their name, BMPs have profound effects on organogenesis processes outside the skeleton.

The search for additional neurotrophic factors will continue in order to provide new members of neurotrophic factor families for use in the diagnosis and treatment of a variety of acute and chronic diseases of the nervous system.

WO 01/72961 discloses a polynucleotide and amino acid sequence for a polypeptide (designated sbg820008-TGFa) comprising 213 amino acids and a truncated form thereof comprising 189 amino acids of the C-terminal end (designated sbg820008-TGFb). Due to close homology to other members of the transforming growth factor (TGF) beta gene family it is assumed that the disclosed polypeptides have corresponding uses in growth control and hence the etiology of cancer, cell differentiation and development, and the following associated diseases are indicated: infection, inflammation, autoimmune disorders, infertility, miscarriage, hematopoietic disorder, wound healing disorder, inflammatory diseases, inflammatory bowel disease, cystic fibrosis, immune deficiency, thrombocytopenia, chronic obstructive pulmonary disease, cf Table III. Furthermore, tissue-specific mRNA expression studies show expression the said polypeptide in a total of ten different tissues, including brain, cf. Table IV. Table V lists a number of diseases related to mRNA expression in each of the ten tissues, and for brain the following diseases are listed: Neurological and psychiatric diseases, including Alzheimers, parasupranuclear palsey, Huntington's disease, myotonic dystrophy, anorexia, depression, schizophrenia, headache, amnesias, anxiety disorders, sleep disorders and multiple sclerosis.

WO 01/92305 discloses a polynucleotide and amino acid sequence for a polypeptide (designated Ztgfβ-10) comprising 212 amino acids and a truncated form thereof comprising 198 amino acids excluding 14 amino acids at the N-terminal end predicted to constitute a signal peptide. Furthermore, WO 01/92305 discloses ten epitope-bearing fragments of the sequence having sizes ranging from 44 to 126 amino acids. The polypeptide may be used to regulate the proliferation, differentiation and apoptosis of neurons, glial cells, lymphocytes, hematopoietic cells and stromal cells.

### SUMMARY OF THE INVENTION

The present invention relates to processed forms of the unprocessed full-length polypeptide disclosed in WO 01/92305. In connection with the present invention the said polypeptide is called NSG3. The said processed forms of NSG3 identified in the present invention include the pro-form of the polypeptide, i.e. excluding the signal peptide, the mature form of the polypeptide and splice variants of the polypeptide. Thus, the present invention has provided the naturally occurring forms of the full-length polypeptide, i.e. the biologically active and relevant forms. The identification of such processed forms of the polypeptide is highly valuable, since it is these forms, which have the biological activity, and hence these forms will have an optimum effect and efficiency in connection with the various uses contemplated by the present invention.

In particular, the present invention relates to the following aspects:

A polypeptide having the amino acid sequence of any of SEQ ID NO: 7, 9, 11, 13, 15, 17, 19, 21 or 23 or a biologically active variant of one of the said sequences having at least 50% identity therewith and comprising between 90 and 188 amino acids and amino acid residues 15, 44, 46, 48, 77, 109 and 111 of SEQ ID NO: 11.

A biologically active polypeptide encoded by a 1) polynucleotide according to any of SEQ ID NO: 6, 8, 10, 12, 14, 16, 18, 20 or 22, 2) a variant of one of the said sequences having at least 30% identity therewith and encoding a polypeptide comprising between 90 and 188 amino acids and amino acid residues 15, 44, 46, 48, 77, 109 and 111 of SEQ ID NO:11, or 3) a polynucleotide, which hybridises to any of SEQ ID NO: 6, 8, 10, 12, 14, 16, 18, 20 or 22 under highly stringent conditions.

A polypeptide for use for treating or preventing a neurodegenerative disease, a cancer, tissue injury, insufficient bone or cartilage growth and maturation or a disease involving muscle, the polypeptide having the amino acid sequence of any of SEQ ID NO: 7, 9, 11, 13, 15, 17, 19, 21 or 23 or a biologically active variant of one of the said sequences having at least 50% identity therewith and comprising between 90 and 188 amino acids and amino acid residues 15, 44, 46, 48, 77, 109 and 111 of SEQ ID NO: 11.

Use of a polypeptide having the amino acid sequence of any of SEQ ID NO: 7, 9, 11, 13, 15, 17, 19, 21 or 23 or a biologically active variant of one of the said sequences having at least 50% identity therewith and comprising between 90 and 188 amino acids and amino acid residues 15, 44, 46, 48, 77, 109 and 111 of SEQ ID NO: 11, for the manufacture of a medicament for treating or preventing a neurodegenerative disease, a cancer, tissue injury, insufficient bone or cartilage growth and maturation or a disease involving muscle.

Use of a polypeptide having the amino acid sequence of any of SEQ ID NO: 3, 5, 7, 9, 11, 13, 15, 17, 19, 21 or 23 or a biologically active variant of one of the said sequences having at least 50% identity therewith and comprising between 90 and 188 amino acids and amino acid residues 15, 44, 46, 48, 77, 109 and 111 of SEQ ID NO: 11, for the manufacture of a medicament for treating an excitotoxic disease.

A pharmaceutical composition comprising as an active substance the polypeptide according to the invention.

A polynucleotide encoding a biologically active polypeptide, wherein the polynucleotide has 1) a sequence according to any of SEQ ID NO: 6, 8, 10, 12, 14, 16, 18, 20 or 22, 2) a variant of one of the said sequences having at least 30% identity therewith and encoding a polypeptide comprising between 90 and 188 amino acids and amino acid residues 15, 44, 46, 48, 77, 109 and 111 of SEQ ID NO: 11, or 3) a polynucleotide, which hybridises to any of SEQ ID NO: 6, 8, 10, 12, 14, 16, 18, 20 or 22 under highly stringent conditions.

A recombinant vector construct comprising 1) a polynucleotide according to any of SEQ ID NO: 6, 8, 10, 12, 14, 16, 18, 20 or 22, 2) a variant of one of the said sequences having at least 30% identity therewith and encoding a polypeptide comprising between 90 and 188 amino acids and amino acid residues 15, 44, 46, 48, 77, 109 and 111 of SEQ ID NO: 11, or 3) a polynucleotide, which hybridises to any of SEQ ID NO: 6, 8, 10, 12, 14, 16, 18, 20 or 22 under highly stringent conditions.

A recombinant host cell comprising the recombinant vector construct according to the invention and/or the polynucleotide of the invention.

A method of producing the polypeptide according to the invention comprising culturing the host cell according to the invention in a culture medium to express the polypeptide, and recovering the polypeptide from the culture medium.

A packaging cell line capable of producing an infective virion comprising the vector construct of the invention.

A pharmaceutical composition comprising the polynucleotide of the invention, the vector construct of the invention, the host cell of the invention or the packaging cell line of the invention.

A method of treating or preventing a neurodegenerative disease, a cancer, tissue injury, insufficient bone or cartilage growth and maturation or a disease involving muscle in an animal comprising administering to the animal an effective amount of the polypeptide according to the invention, the polynucleotide of the invention, the vector construct of the invention, the host cell of the invention or the packaging cell line of the invention.

A method of treating or preventing a excitotoxic disease in an animal comprising administering to the animal an effective amount of

a polypeptide having the amino acid sequence of any of SEQ ID NO: 3 or 5 or a biologically active variant of one of the said sequences having at least 50% identity therewith and comprising between 90 and 188 amino acids and amino acid residues 15, 44, 46, 48, 77, 109 and 111 of SEQ ID NO: 11;

a polynucleotide encoding a biologically active polypeptide, wherein the polynucleotide has 1) a sequence according to any of SEQ ID NO: 2 or 4, 2) a variant of one of the said sequences having at least 30% identity therewith and encoding a polypeptide comprising between 90 and 188 amino acids and amino acid residues 15, 44, 46, 48, 77, 109 and 111 of the SEQ ID NO: 11, or 3) a polynucleotide, which hybridises to any of SEQ ID NO: 2 or 4 under highly stringent conditions;

a recombinant vector construct comprising the said polynucleotide;

a recombinant host cell comprising the said recombinant vector construct and/or the said polynucleotide; or

a packaging cell line capable of producing an infective virion comprising the said vector construct.

### DETAILED DISCLOSURE OF THE INVENTION

Novel NSG3 Polypeptides

In its first aspect, the invention provides novel processed forms of the polypeptide having the amino acid sequence presented as SEQ ID NO: 3, and related polypeptides. In the context of this invention, the polypeptide having the amino acid sequence presented as SEQ ID NO: 3 is designated Neuronal Survival and Growth Factor 3 (NSG3).

Based on a Clustal X (1.64b) multiple sequence alignment, SEQ ID NO: 15 was aligned with other members of the TGF-β super family, and the result is shown in FIG. 1. SEQ ID NO: 15 represents the predicted core sequence of NSG3 plus one additional amino acid at each end. As will appear from FIG. 1, seven amino acid residues are conserved in all members of the TGF-β super family, viz. Cystein at positions 15, 44, 48, 77, 109 and 111 and Glysine at position 46 of the mature form of NSG3 (SEQ ID NO: 11). It is believed that these seven residues are all essential for the folding and hence the function of the polypeptide. Based on the structure of other members of the TGF-β super family, NSG3 comprise the following disulfide bridges: Cys15-Cys77, Cys44-Cys109 and Cys48-Cys111.

A phylogenetic tree based on this multiple sequence alignment is presented in FIG. 2. This plates NSG3 close to the Glial Cell Derived Neurotrophic Favor (GDNF) sub-family of neurotrophic factors. The closest matches in terms of identity to the NSG3 mature polypeptides are found in Growth and Differentiation Factor 3 (GDF) (Homo sapiens) and inhibin/activin (Oryzias latipes)

The predicted core sequence of NSG-3 plus one additional amino acid at each end (99 residues; SEQ ID NO. 15) was compared using the BLAST program (Altshul, S. F., Gish, W., Miller, W., Myers, E. W. & Lipman, D. J. (1990) “Basic local alignment search tool.” J. Mol. Biol. 215.403410.) against the set of the corresponding partial sequences of the TGF-beta family members. The results are shown in Table 1 below.

TABLE 1
NSG-3 homology to other TGF-beta members
The percentage of identical and positive matches is summarized
in the table below.

| | | Identities/ | | Similarities/ |
|---|---|---|---|---|
| TGF-beta | | alignment | | alignment |
| family protein | % Identity | length | % Similarily | length |
| GDF-3 | 29 | 31/104 | 46 | 49/104 |
| Inhibin beta-B | 28 | 30/106 | 45 | 49/106 |
| BMP-8 | 24 | 26/107 | 39 | 43/107 |
| GDNF | 21 | 16/73 | 33 | 25/73 |

in Table 1 , “% Similarity” was defined as follows: Amino acid substitutions having a positive score in the Blosum62 amino acid substitution matrix (Henikoff, S & Henikoff, J. G. (1992). Proc. Natl. Acad. Sci. USA 89:10915-19) were allowed and included in the similarity measure. The following substitutions for each amino acid have positive scores:

| Amino | May be replaced by |
|---|---|
| acid | amino acid(s) |
| P | None |
| C | None |
| G | None |
| A | S |
| S | T A N |
| T | S |
| N | D H S |
| B | D N E |
| D | B E N Z |
| E | Z D Q K B |
| Z | E Q D K |
| Q | Z E K R |
| K | R Q E Z |
| R | K Q |
| H | Y N |
| W | Y F |
| Y | F W H |
| F | Y W |
| M | L I V |
| L | I M V |
| I | V L M |
| V | I L M |

Preferably, the variant of SEQ ID NO: 7, 9, 11, 13, 15, 17, 19, 21 or 23 has at least 60%, more preferably 70%, more preferably 80%, more preferably 90%, more preferably 96% and most preferably 98% identity therewith.

Preferably, the polypeptide of the invention has 97-180, more preferably 97-140, and most preferably 97-112 amino acids. In a particular preferred embodiment of the invention, the polypeptide has the amino acid sequence of SEQ ID NO:11, i.e. the mature form of the protein, or a biologically active variant thereof having at least 90% identity therewith.

It is known that some growth factor polypeptides are secreted without cleavage of a signal peptide. Fibroblast growth factor (FGF)-9 is a neurotrophic polypeptide expressed in the brain. The mechanism for its secretion from expressing cells is unclear, because its primary structure lacks a cleavable signal sequence. Miyakawa et al. (J Biol Chem 1999 Oct. 8;274(41):29352-7) found two hydrophobic domains, located at the N terminus and at the centre of the FGF-9 primary structure. Examination of various point mutants revealed that local hydrophobicity of the central hydrophobic domain, but not the N terminus, was crucial for translocation.

Ciliary neurotrophic factor (CNTF) is expressed in glial cells within the central and peripheral nervous systems. CNTF itself lacks a classical signal peptide sequence of a secreted protein, but is thought to convey its cytoprotective effects after release from adult glial cells by some mechanism induced by injury. (Sleeman M W et al. Pharm Acta Helv 2000 March; 74(2-3): 265-72).

The observed splice variant polypeptides of the present invention, encoded by the nucleotide sequences presented as SEQ ID NO: 4 and 6, do not have a cleavable signal peptide but may still be secreted by some other process.

Members of the TGF-beta family of growth factors e.g. GDNF are known to be biologically active in a truncated form. The group of biologically active truncated forms of NSG3 comprise at its extreme the form delimited by the first and the last of the seven amino acid residues conserved in all members of the TGF-⊖ super family, i.e. the form comprising amino acids 15-111 of the mature form of NSG3 (SEQ ID NO:11) and being truncated at both its C- and N-termini.

Novel NSG2 Polypeptides

SEQ ID NO: 16-24 relate to novel processed forms of the polypeptide having the amino acid sequence presented as SEQ ID NO: 24, and related polypeptides. In the context of this invention, the polypeptide having the amino acid sequence presented as SEQ ID NO: 24 is designated Neuronal Survival and Growth Factor 2 (NSG2). NSG2 is a natural occurring variant of NSG3, and the polynucleotide of SEQ ID NO: 24 comprise a stop codon in the section encoding the propeptide at position 169-171 (TGA). SEQ ID NO: 16 is a mutated polynucleotide sequence encoding a variant proform of NSG2, wherein the stop codon has been replaced by the corresponding sequence of NSG3, i.e. CAA encoding Gln. SEQ ID NO: 19 is the projected mature form of NSG2, and SEQ ID NO: 21 and 23 are a first and a second form of NSG2 truncated at both ends comprising the core of NSG2, i.e. the partial sequence from the first to the last of the seven amino acids conserved in all members of the TGF-β super family, and the core of NSG2 and one additional amino acid at each end, respectively. In NSG2, the seven amino acid residues conserved in all members of the TGF-β super family are located as follows: Cystein at positions 15, 44, 48, 82, 114 and 116 and Glysine at position 46 of the mature form of NSG2 (SEQ ID NO: 19). Based on the structure of other members of the TGF-β super family, NSG2 comprise the following disulfide bridges: Cys15-Cys82, Cys44-Cys114 and Cys48-Cys116.

In a preferred embodiment of the invention the variant of the polypeptide of the invention is a hybrid between any of SEQ ID NO: 7, 9, 11, 13 or 15 and any of SEQ ID NO: 17, 19, 21 or 23.

Amino Acid Sequence Identity

The polypeptide identity referred to above of the polypeptide of the invention is determined as the degree of identity between two sequences indicating a derivation of the first sequence from the second.

In connection with the present invention, the identity is defined as the identity determined by means of computer programs known in the art as GAP provided in the GCG program package [Needleman, S. B. and Wunsch, C. D., Journal of Molecular Biology, 1970 48 443-453] using GAP with the following settings for polypeptide sequence comparison: GAP creation penalty of 3.0 and GAP extension penalty of 0.1.

Based on the identity determination it is confirmed that the polypeptide of the invention, belonging to the TGF-β superfamily, is related to the GDNF subfamily, but represents a distinct member of this subfamily.

The polypeptide of the invention may be isolated from mammalian cells, preferably from a human cell, more preferred from human brain tissue.

The Polynucleotides

In another aspect the invention provides polynucleotides useful for expression of the polypeptides of the invention.

The polynucleotide of the invention may preferably be obtained by cloning procedures, e.g. as described in “Current Protocols in Molecular Biology” (available from John Wiley & Sons, Inc.). In a preferred embodiment, the polynucleotide is cloned from, or produced on the basis of a cDNA library of the human brain.

In a preferred embodiment, the polynucleotide of the invention has a nucleic acid (DNA) sequence capable of hybridising under high stringency conditions with any one of the polynucleotide sequences presented as SEQ ID NOS: 6, 8, 10, 12, 14, 16, 18, 20 or 22, its complementary strand, or a sub-sequence thereof.

In another preferred embodiment, the isolated polynucleotide of the invention has a nucleic acid (DNA) sequence having at least 50%, preferably at least 60%, more preferably at least 70%, preferably at least 80%, more preferably at least 90%, more preferably at least 95% and most preferably at least 98% identity to any one of the polynucleotide sequences presented as SEQ ID NO: 6, 8, 10, 12, 14, 16, 18, 20 or 22.

In a preferred embodiment, the polynucleotide has the DNA sequence presented as SEQ ID NO: 6, 8, 10, 12, 14, 16, 18, 20 or 22, most preferably SEQ ID NO:11.

Identity of DNA Sequences

The DNA sequence identity referred to above is determined as the degree of identity between two sequences indicating a derivation of the first sequence from the second. In connection with the present invention, the identity is defined as the identity determined by means of computer programs known in the art as GAP provided in the GCG program package (Needleman, S. B. and Wunsch, C. D., (1970), Journal of Molecular Biology, 48, 443-453) using GAP with the following settings for DNA sequence comparison: GAP creation penalty of 5.0 and GAP extension penalty of 0.3.

Hybridisation

In connection with the present invention the expression “highly stringent conditions” are defined as follows: The experimental conditions for determining hybridisation at high stringency between a nucleotide probe and a homologous DNA or RNA sequence involves presoaking of the filter containing the DNA fragments or RNA to hybridise in 5×SSC (Sodium chloride/Sodium citrate; cf. Sambrook et al. Molecular Cloning: A Laboratory Manual, Cold Spring Harbor Lab.; Cold Spring Harbor, N.Y. 1989) for 10 minutes, and prehybridization of the filter in a solution of 5×SSC, 5× Denhardt's solution (cf. Sambrook et al. Op cit.), 0.5% SDS and 100 μg/ml of denatured sonicated salmon sperm DNA (cf. Sambrook et al. Op cit.), followed by hybridisation in the same solution containing a concentration of 10 ng/ml of a random-primed (Feinberg, A. P. and Vogelstein, B., Anal. Biochem. 1983 132:6-13), ³²P-dCTP-labeled (specific activity>1×10⁹ cpm/μg) probe for 12 hours at approx. 45° C. The filter is then washed twice for 30 minutes in 2×SSC, 0.5% SDS at least 70° C., and even more preferably at least 75° C.

Molecules to which the oligonucleotide probe hybridises under these conditions may be detected using an x-ray film.

Recombinant Expression Vectors

In a further aspect the invention provides a recombinant expression and transfection vector construct comprising the polynucleotide of the invention.

The recombinant expression vector of the invention may be any suitable eukaryotic expression vector. Preferred recombinant expression vectors are pTEJ-8 (FEBS Lett. 1990 267 289-294) and pcDNA-3 [available from Invitrogen].

Host Cells

In a yet further aspect the invention provides a recombinant host cell comprising the isolated polynucleotide sequence of the invention, and/or or a recombinant expression vector of the invention.

The host cell of the invention may preferably be a eukaryotic cell, in particular a human cell, or a fungal cell, such as a yeast cell or a filamentous fungal cell. Preferred mammalian cell are CHO, HEK293, COS, PC12, HiB5, RN33b cell lines and human neural stem cells.

In a more preferred embodiment the isolated polynucleotide sequence of the invention, and/or or a recombinant expression vector of the invention are transfected in a mammalian host cell, an astrocyte cell, a T-cell, a haematopoietic stem cell, a non-dividing cell, or a cerebral endothelial cell, comprising at least one DNA molecule capable of mediating cellular immortalization and/or transformation.

The host cell may also be a prokaryotic cell such as E. coli.

Method of Producing the Polypeptides

In another aspect the present invention provides a method of producing an isolated polypeptide of the invention. In the method of the invention a suitable host cell, which has been transformed with a DNA sequence encoding the polypeptide, is cultured under conditions permitting the production of the polypeptide, followed by recovery of the polypeptide from the culture medium.

In a further aspect the present invention provides a method of producing an isolated polypeptide of the invention wherein the coding sequence for the signal peptide, and/or the pro-peptide are replaced by a polynucleotide coding for the signal peptide and/or the pro-peptide of a further growth factor polypeptide. In a preferred embodiment the further growth factor polypeptide is selected from GDNF, Neublastin, Persephin, Neurturin or NSG2.

Pharmaceutical Compositions

In another aspect the invention provides novel pharmaceutical compositions comprising a therapeutically effective amount of the polypeptide of the invention.

For use in therapy the polypeptide of the invention may be administered in any convenient form. In a preferred embodiment, the polypeptide of the invention is incorporated into a pharmaceutical composition together with one or more adjuvants, excipients, carriers and/or diluents, and the pharmaceutical composition prepared by the skilled person using conventional methods known in the art.

Methods of Treatment

In yet another aspect the invention relates to a method of treating or alleviating a disorder or disease of a living animal body, including a human, which disorder or disease is responsive to the activity of neurotrophic agents.

In a preferred embodiment of the method of the invention, the disease or disorder is a neurodegenerative disease involving lesioned and traumatic neurons, such as traumatic lesions of peripheral nerves, the medulla, and/or the spinal cord, cerebral ischaemic neuronal damage, neuropathy and especially peripheral neuropathy, Alzheimer's disease, Huntington's disease, Parkinson's disease, amyotrophic lateral sclerosis or any other neurodegenerative disease, and memory impairment connected to dementia. In a particularly preferred embodiment the neurodegenerative disease is an excitotoxic disease. Preferably, the excitotoxic disease is selected from the group consisting of ischaemia, epilepsy and trauma due to injury, cardiac arrest or stroke. In connection with the present invention, the term “excitotoxic disease” is defined as follows: A condition of interrupted or impaired blood supply to the brain leading to ischaemia (lack of both oxygen and glucose), hypoxia (lack of oxygen) or hypoglycemia (lack of glucose).

In a still further aspect, the disease or disorder treated or prevented in accordance with the invention is one related to insufficient bone or cartilage growth and maturation, such as osteoporosis, osteohalisteresis, and osteomalacia.

Also, the polypeptides of the invention are useful in repair of tissue injury caused by e.g. trauma or burns.

The polypeptides of the invention also have applications in treating disease processes involving muscle, such as in musculodegenerative diseases or in tissue repair due to trauma. In this regard, many other members of the TGF-beta family are also important mediators of tissue repair.

The polypeptides of the invention also have applications in the treatment of various types of cancer. Several known members of this family can function as tumour suppressors. For example, inhibin alpha has been shown to suppress the development of both gonadal and adrenal tumours. Similarly, MIS has been shown to inhibit the growth of human endometrial and ovarian tumours in nude mice.

Another object of the present invention is to provide a method for the prevention of the degenerative changes connected with the above diseases and disorders.

In a preferred embodiment, the gene encoding the polypeptide of the invention is transfected into a suitable cell line, e.g. into an immortalized rat neural stem cell line like HiB5 and RN33b, or into a human immortalized neural stem cell line, and the resulting cell line is implanted in the brain of a living body, including a human, to secrete the therapeutic polypeptide of the invention in the CNS, e.g. using the expression vectors described in International Patent Application WO 98/32869.

Other Applications of the Polypeptides of the Invention

The polypeptides of the invention are suitable for use as an in-vitro supplement for the growth and/or differentiation of stem cells and progenitor cells.

The polypeptides of the invention are suitable for generating therapeutic or diagnostic antibodies.

### BRIEF DESCRIPTION OF THE DRAWINGS

FIG. 1 shows a Clustal X (1.64b) multiple sequence alignment of NSG3 and NSG2 with the other members of the TGF-β super family.

FIG. 2 shows a phylogenetic tree based on the multiple sequence alignment presented in FIG. 1.

FIG. 3 shows a schematic presentation of the NSG3 primary genomic transcript (SEQ ID NO: 1) and the parts constituting full-length NSG3 (SEQ ID NO: 2) and two splice variants (SEQ ID NO: 4 and 6).

FIG. 4 is a photograph of a gel showing the expression of a 281 bp fragment of NSG3 in a number of tissues.

FIG. 5 shows a diagram of NSG3 expression in a number of tissues.

FIG. 6 shows a diagram of the experimental protocol used in Example 5.

FIGS. 7A and B show a diagram of the neuroprotective effect of NSG3 on hippocampal neurons against the excitotoxic effects of NMDA.

FIG. 8 shows the activation effect of NSG3 on various receptors of the central nervous system.

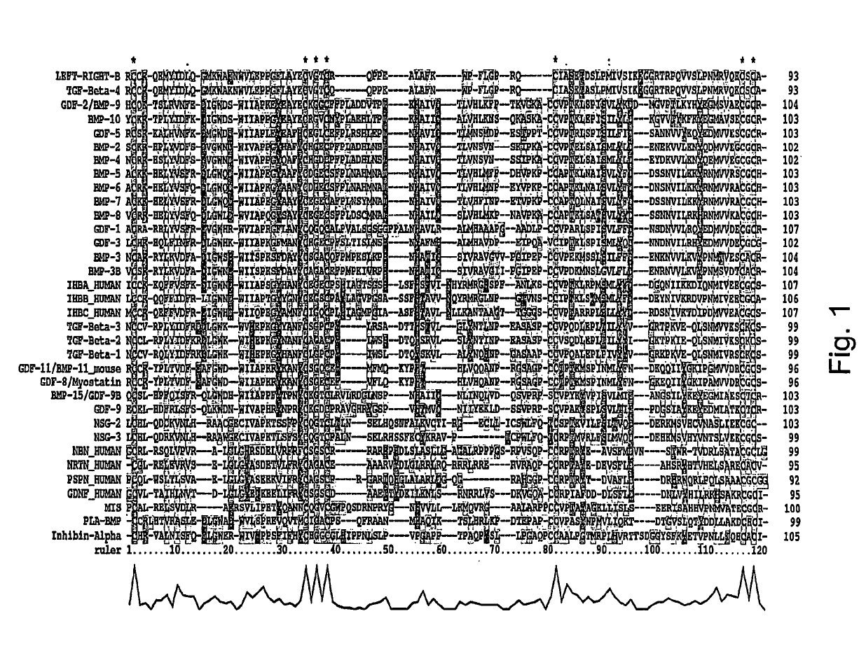

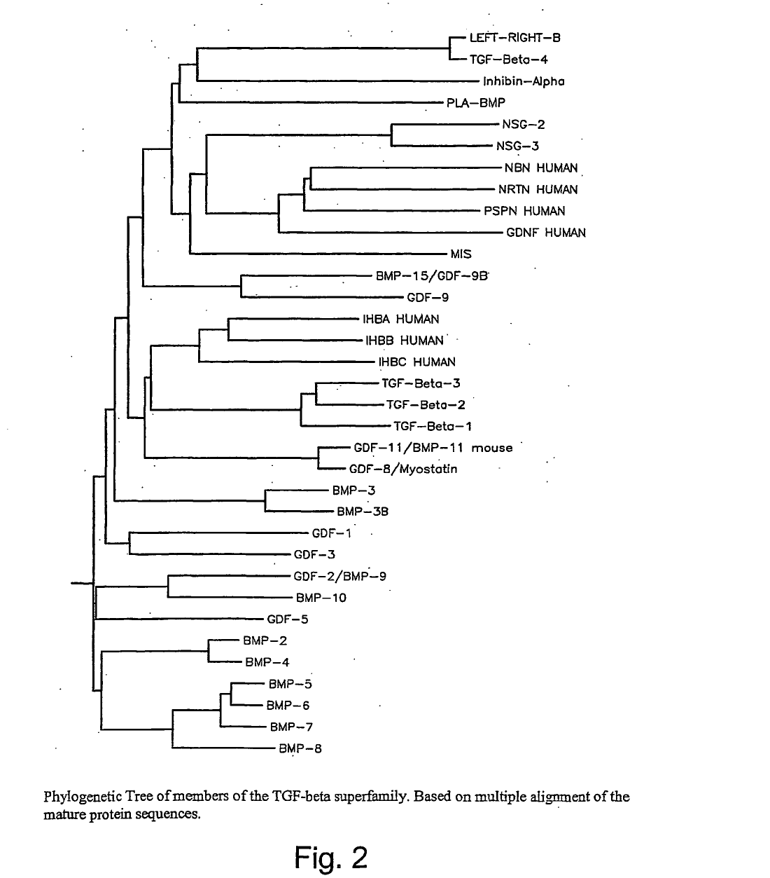

### LIST OF SEQUENCES

SEQ ID NO: 1 is the sequence of NSG3 primary genomic transcript (1995 bp)

SEQ ID NO: 2 is the sequence of cDNA for full-length NSG3 (639 bp; 212 aa)

SEQ ID NO: 3 is the amino acid sequence corresponding to SEQ ID NO: 2

SEQ ID NO: 4 is the sequence of one splice variant of NSG3 (585 bp; 194 aa)

SEQ ID NO: 5 is the amino acid sequence corresponding to SEQ ID NO: 4

SEQ ID NO: 6 is the sequence of a second splice variant of NSG3 (423 bp; 140 aa)

SEQ ID NO: 7 is the amino acid sequence corresponding to SEQ ID NO: 6

SEQ ID NO: 8 is the sequence of NSG3 pro-protein (540 bp; 179 aa)

SEQ ID NO: 9 is the amino acid sequence corresponding to SEQ ID NO: 8

SEQ ID NO: 10 is the sequence of the mature form of NSG3 (339 bp; 112 aa)

SEQ ID NO: 11 is the amino acid sequence corresponding to SEQ ID NO: 10

SEQ ID NO: 12 is the sequence of a first fragment of NSG3 truncated at both ends (291 bp; 97 aa)

SEQ ID NO: 13 is the amino acid sequence corresponding to SEQ ID NO: 12

SEQ ID NO: 14 is the sequence of a second fragment of NSG3 truncated at both ends (300 bp; 99 aa)

SEQ ID NO: 15 is the amino acid sequence corresponding to SEQ ID NO: 14

SEQ ID NO: 16 is the sequence of a mutated NSG2 pro-protein (555 bp; 184 aa)

SEQ ID NO: 17 is the amino acid sequence corresponding to SEQ ID NO: 16

SEQ ID NO: 18 is the sequence of the projected mature form of NSG2 (354 bp; 117 aa)

SEQ ID NO: 19 is the amino acid sequence corresponding to SEQ ID NO: 18

SEQ ID NO: 20 is the sequence of a first fragment of NSG2 truncated at both ends (306 bp; 102 aa)

SEQ ID NO: 21 is the amino acid sequence corresponding to SEQ ID NO: 20

SEQ ID NO: 22 is the sequence of a second fragment of NSG2 truncated at both ends (315 bp; 104 aa)

SEQ ID NO: 23 is the amino acid sequence corresponding to SEQ ID NO: 22

SEQ ID NO: 24 is the sequence of cDNA for full-length NSG2 (654 bp; 216 aa)

bp: base pair

aa: amino acid

In connection with the present invention the term “full-length protein” means the polypeptide comprising the signal peptide, the pro-peptide and the mature peptide.

In connection with the present invention the term “pro-protein” means the polypeptide comprising the pro-peptide and the mature form of the sequence.

### EXAMPLES

The following examples are provided for illustrative purposes only, and are not intended to be limiting

### Example 1

### Cloning of Genomic NSG-3

Based on the genomic sequence of a human clone identified in Genbank (accession number AC008940) a single primer set was designed using Oligo software (National Biosciences). This primer set, illustrated below was designed to amplify putative full length NSG-3 genomic sequence.

| | | |
|---|---|---|
| Primer set No. 1 | | |
| 5′-GATGCTGCCTTCCCACATAA | (Sense) | (PRIMER: 1) |
| AT-3′ | | |
| 5′-GTTACTGCCATAATGCCAACCT | (Antisense) | (PRIMER: 2) |
| TCT-3′ | | |

Using this primer set a 1995 bp DNA fragment was amplified from genomic DNA.

PCR Protocol:

PCR was performed using Platinum Taq thermostable polymerase (GIBCO BRL) with standard buffer. The PCR reaction mixture was supplemented with 1.5 mM MgCl₂ and loading buffer to a final concentration of 12% sucrose, 3 mM Cresol red. The total PCR reaction volume was 25 μl. Thermocycling was performed in a PTC-225 DNA engine thermocycler (MJ Research) using a cycling profile consisting of a 5 minute pre-denaturation step at 94° C., followed by 40 three-step cycles at 94° C. for 20 seconds, 60° C. for 15 seconds and extension at 68° C. for 90 seconds respectively. Thermocycling was terminated by a 5 minute incubation at 68° C. PCR products were loaded onto a 1% agarose gel (FMC) and photographed.

The 1995 bp PCR product amplified from human genomic DNA was cloned into the pCR4-TOPO vector using the TOPO TA cloning kit from Invitrogen. The vector was transformed into competent cells supplied with the cloning kit. The insert was sequenced using the following 3 sequencing primers:

| | |
|---|---|
| NSG3 421-440: | 5′-GCTGCTGGATCAGGGCTGTT-3′ |
| M13 Reverse Primer: | 5′-CAGGAAACAGCTATGAC-3′ |
| M13 Forward Primer: | 5′-GTTTTCCCAGTCACGA-3′ |

### Example 2

### Expression Analysis of NSG-3

To investigate the expression pattern of the NSG-3 gene, a panel of fetal and adult cDNA was subjected to RT-PCR analysis using techniques described below.

Method of Detecting NSG-3 RNA Expression by RT-PCR:

Using a primer set located within predicted exon 2 of the NSG-3 gene as described below. This and subsequent primer sets were designed using Oligo software (National Biosciences)

| | | |
|---|---|---|
| Primer set No. 2 | | |
| 5′-ATGCCAGCCTGAATGAA-3′ | (Sense) | (PRIMER: 3) |
| 5′-CTTGGGTGCAACAATACACT-3′ | (Antisense) | (PRIMER: 4) |

Using this primer set, a 281 bp fragment was amplified using human genomic and cDNA. The primer set was used to RT-PCR amplify a DNA fragment from a panel of human cDNAs composed of fetal (lung, arm, liver, intestine) and adult (testis, lung, kidney, brain, heart, adrenal gland and placenta) cDNA. Fetal tissues were obtained from an aborted fetus, and total RNA was extracted using standard techniques (Chomczynski P. and Sacchi N. (1987) Anal. Biochem., 162:156-159). Adult samples were purchased as total RNA (Clontech). Furthermore, poly-A RNA from fetal and adult brain was purchased (Clontech) and included in the RT-PCR analysis.

PCR Protocol:

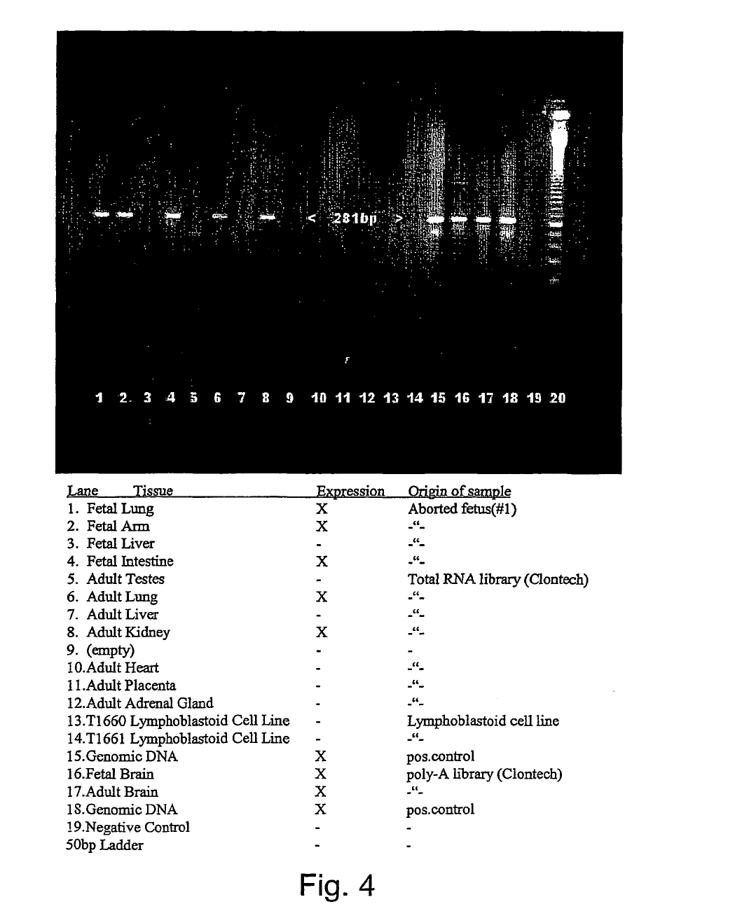

PCR was performed using Taq thermostable polymerase (Amersham Pharmacia Biotech) with standard buffer. The PCR reaction mixture was supplemented with loading buffer to a final concentration of 12% sucrose, 3 mM Cresol red. The total PCR reaction volume was 15 μl. Thermocycling was performed in a PTC-225 DNA engine thermocycler (MJ Research) using a cycling profile consisting of a 5 minute pre-denaturation step at 94° C., followed by 40 three-step cycles at 94° C. for 15 seconds, 57° C. for 15 seconds and extension at 72° C. for 20 seconds respectively. Thermocycling was terminated by a 5 minute incubation at 72° C. PCR products were loaded onto a 2% agarose gel (FMC Bioproducts, Rockland, Me.) and photographed. As illustrated in FIG. 4, the 281 bp NSG-3 specific fragment was observed in fetal lung, arm and intestine. In adult tissues expression of the 281 bp NSG-3 fragment was observed in lung and kidney. Furthermore, expression of the 281 bp NSG-3 was observed in both fetal and adult poly-A RNA. To verify that this fragment represented NSG-3 sequences, the 281 bp fragment was cloned into pCRII (Invitrogen) and sequenced using the following 2 sequencing primers:

| | |
|---|---|
| M13 Reverse Primer: | 5′-CAGGAAACAGCTATGAC-3′ |
| M13 Forward Primer: | 5′-GTTTTCCCAGTCACGA-3′ |

A second primer set, located in putative exon 3 of the NSG-3 coding sequence was designed:

| | | |
|---|---|---|
| Primer set No. 3 | | |
| 5′-CCACCATGGTCAGACTCT | (Sense) | (PRIMER: 5) |
| 5′-CTCATCTTGTGTTCGTCATC | (Antisense) | (PRIMER: 6) |

Using this primer set, a 55 bp fragment of the expected length was amplified in a RT-PCR reaction using template obtained from adult and fetal brain poly-A RNA, indicating that both exon 2 and exon 3 of the NSG-3 coding sequence was expressed in these tissues.

PCR Protocol:

PCR was performed using Taq thermostable polymerase (Amersham Pharmacia Biotech) with standard buffer. The PCR reaction mixture was supplemented with loading buffer to a final concentration of 12% sucrose, 3 mM Cresol red. The total PCR reaction volume was 15 μl. Thermocycling was performed in a PTC-225 DNA engine thermocycler (MJ Research) using a cycling profile consisting of a 5 minute pre-denaturation step at 94° C., followed by 40 three-step cycles at 94° C. for 15 seconds, 57° C. for 15 seconds and extension at 72° C. for 20 seconds respectively. Thermocycling was terminated by a 5 minute incubation at 72° C. PCR products were loaded onto a 2% agarose gel (FMC) and photographed.

### Example 3

### Method of Analyzing Partial NSG-3 Genomic Structure and mRNA Splicing by RT-PCR

In order to investigate if the apparent expression data obtained above represented a coherent mRNA encoding the entire part of the putative mature peptide of NSG-3, a new primer set was designed. The sequences of these primers were located in exon 2 and exon 3 respectively, thus potentially spanning putative intron 2.

| | | |
|---|---|---|
| Primer set No. 4 | | |
| 5′-GCCACCTCTGTTTCACCTGCCT | (Sense) | (PRIMER: 7) |
| TAT-3′ | | |
| 5′-TTGTGTTCGTCATCCTGGACCA | (Antisense) | (PRIMER: 8) |
| TC-3′ | | |

Using primer set no. 4 a 374 bp fragment was amplified from adult brain cDNA, representing the correct predicted splicing of exon 2 and 3. The 374 bp PCR product was cloned into TOPO TA cloning vector PCR2.1 and sequenced to verify that this PCR product represented NSG-3 using the following sequencing primers:

| | |
|---|---|
| M13 Reverse Primer: | 5′-CAGGAAACAGCTATGAC-3′ |
| M13 Forward Primer: | 5′-GTTTTCCCAGTCACGA-3′ |

PCR Protocol:

PCR was performed using Taq thermostable polymerase (Amersham Pharmacia Biotech) with standard buffer. The PCR reaction mixture was supplemented with loading buffer to a final concentration of 12% sucrose, 3 mM Cresol red The total PCR reaction volume was 15 μl. Thermocycling was performed in a PTC-225 DNA engine thermocycler (MJ Research) using a cycling profile consisting of a 5 minute pre-denaturation step at 94° C., followed by 40 three-step cycles at 96° C. for 15 seconds, 65° C. for 15 seconds and extension at 72° C. for 45 seconds respectively. Thermocycling was terminated by 5 minutes incubation at 72° C. PCR products were loaded onto a 2% agarose gel (FMC) and photographed.

Method of Quantitation

NSG-3 RNA Expression by Lightcycler RT-PCR:

A quantitative assay for NSG-3 expression was established using a real-time thermocycler (MJ-Research) and primer set composed of PRIMER 3 and 4. A PCR product was prepared by amplification of human genomic DNA to be used for the preparation of a standard curve for lightcycler quantitation. The PCR product was gel purified, precipitated and resuspended in a solution containing 60 ng/μl tRNA. Following resuspension, the concentration of the PCR product was determined spectrophotometrically. A serial dilution was prepared and subjected to PCR amplification using a real-time thermocycler, using the same PCR protocol as described below. For normalization purposes, a PCR product of GAPDH was produced using primers:

| | |
|---|---|
| GAPDH A: | 5′-ACAGTCCATGCCATCACTGCC-3′ |
| GAPDH B: | 5′-GCCTGCTTCACCACCTTCTTG-3′ |

This PCR product was gel purified, precipitated and resuspended in water. A serial dilution was prepared and subjected to amplification using the PCR protocol described below.

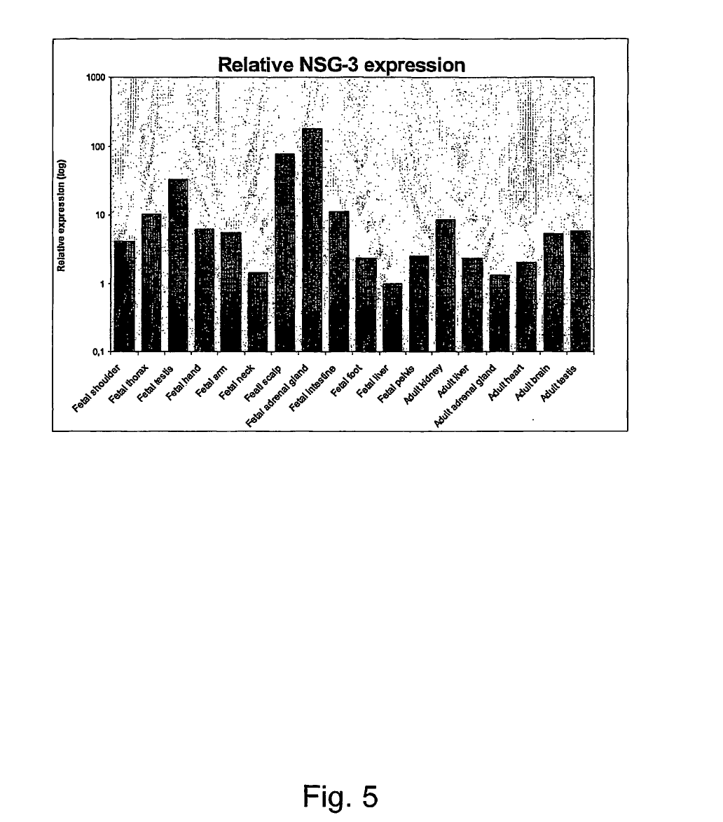

New cDNA was prepared from fetal (shoulder, thorax, testis, hand, arm, neck scalp, adrenal gland, intestine, foot, liver and pelvis/femur) and adult (kidney, liver, adrenal gland, heart, lung, brain and testis) total RNA samples. Adult total RNA was obtained from Clontech The newly synthesized cDNA was used in a PCR reaction as described below. These data show that NSG-3 expression is most prominent in the fetal adrenal gland and that expression in the adult is most prominent in the kidney (see Table 2 below and FIG. 5).

TABLE 2

| | |
|---|---|
| Tissue | Fold expression* |

| | | |
|---|---|---|
| Fetal tissues | Shoulder | 4.15 |
| | Thorax | 4.18 |
| | Testis | 32.78 |
| | Hand | 6.21 |
| | Arm | 5.46 |
| | Neck | 1.42 |
| | Scalp | 77.77 |
| | Adrenal gland | 176.19 |
| | Intestine | 11.23 |
| | Foot | 2.32 |
| | Liver | 1 |
| | Pelvis | 2.52 |
| Adult tissues | Kidney | 8.45 |
| | Liver | 2.36 |
| | Adrenal gland | 1.33 |
| | Heart | 2.04 |
| | Brain | 5.31 |
| | Testis | 5.83 |

*Expression levels indicated relative to level in fetal liver (set at 1.0)

PCR Protocol:

PCR for the NSG-3 standard curve was performed in quadruplicate and for the tissues, PCR was performed in duplicate, using the lightcycler PCR kit (Roche). The PCR reaction mixture was supplemented with 2 mM MgCl₂ in a total reaction volume of 15 μl. Thermocycling was performed in the lightcycler (Roche) using a cycling profile consisting of a 15 minute pre-denaturation step at 94° C., followed by 45 three-step cycles at 94° C. for 20 seconds, 57° C. for 20 seconds and extension at 72° C. for 20 seconds respectively. Following thermocycling, the temperature was lowered to 57° C. and the temperature was then slowly raised from 57° C. to 95° C. with continuous data acquisition to prepare melting curves for the produced fragments. A separate PCR reaction in duplicate was was performed on a serial dilution of the purified GAPDH fragment and on tissue cDNAs using the the real-time thermocycler. PCR thermocycling protocol and reaction conditions was identical to that for NSG-3.

Data obtained from the serial dilutions of NSG-3 and GAPDH fragments were used to produce standard curves from these genes. From the standard curve of GAPDH, expression levels of GAPDH in the tissues analysed were calculated. Assuming that the calculated expression values of GAPDH should be identical, NSG-3 expression data was adjusted. From the NSG-3 standard curve, expression levels of NSG-3 was calculated for the tissues analyzed. Following GAPDH adjusting and expression level calculation, NSG-3 data was normalized against the lowest expression level (fetal liver) (shown graphically in FIG. 5).

### Example 4

### Method for Production of NsG-3 in a Mammalian Cell Line

In order to study the biological effects of NsG-3 mammalian cell lines expressing NsG-3 were generated as described below.

Expression Vectors

A genomic sequence (SEQ ID NO: 1) corresponding to the primary NsG-3 transcript was amplified by PCR using Primer set No. 1 and inserted into the pUbi1z (Ubiquitin promoter) eukaryotic transfection vector resulting in pUbi1Z-NsG-3-g.

The pUbi1Z vector was generated by cloning the human UbC (ubiquitin) promoter into a modified version of pcDNA3.1/Zeo. The unmodified pcDNA3.1/Zeo is commercially available (Invitrogen). The modified pcDNA3.1/Zeo is smaller than the parent vector, because the ampicillin gene (from position 3933 to 5015) and a sequence from position 2838 to 3134 were removed. In this modified version of pcDNA3.1/Zeo, the CMV promoter was replaced with the UbC promoter from pTEJ-8 (Johansen T E et al. (1990), FEBS Lett. 267:289-294), resulting in pUbi1Z.

Mammalian Cell Expression

The linearised pUbi1Z-NsG-3-g vector construct was transfected into HiB5 cells using Lipofectamin Plus. HiB5 is an immortalised rat neural cell line (Renfranz P J et al. (1991), Cell, 66:713-729). After 48 hrs, selection was started in 100 ¼ g Zeocin/ml. RNA was extracted from pools of clones and cDNA was synthesized to perform RT-PCR tests. One clone, named HiB5-NsG3g-2, was used for further studies.

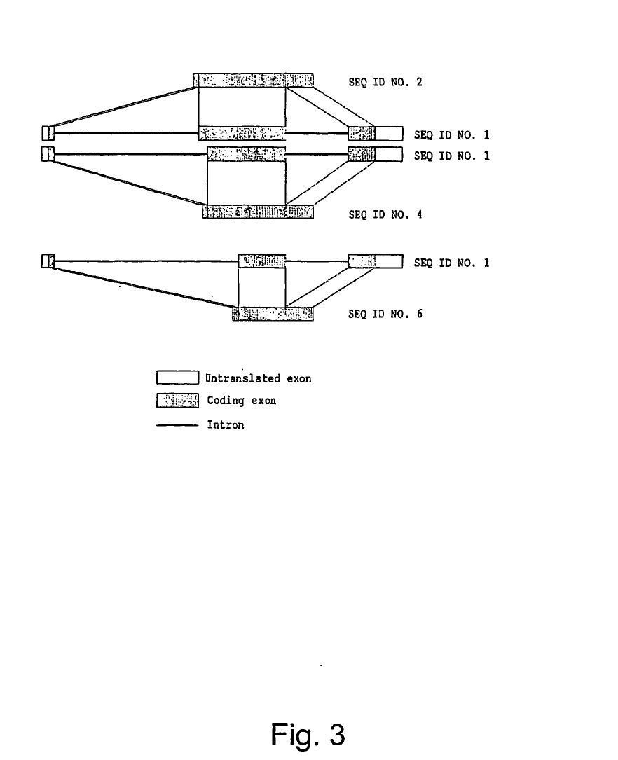

The RT-PCR tests showed a major product of 600 bp and additional products of approximately 700, 850 and 1000 bp. These products were cloned into the pCR II-TOPO vector and sequenced. The 1000 bp product represents a splice variant with an internal stop codon in exon 2 of NsG-3. The fragment of approx. 600 bp contained the claimed SEQ ID NO: 6, the fragment of approx. 700 bp. contained SEQ ID NO: 4 and the fragment of approx. 850 bp contained SEQ ID NO: 2. The different splice variants are shown schematically in FIG. 3.

The fragment of approx. 850 bp, containing the full-length 639 bp cDNA for NsG-3 represented by SEQ ID NO: 2, was excised from an agarose gel and cloned into the pUbi1Z vector resulting in the pUbi1Z-NsG-3-c vector. The vector pUbi1Z-NsG-3-c was used for transfection into HiB5 cells, which resulted in the selection of the clone designated HiB5-NsG3c4, which was used for further studies.

### Example 5

### Method for Assessing the Neuroprotective Effect of NsG-3

In order to assess the neuroprotective effect of NsG-3 on primary hippocampal neurons against the excitotoxic effects of the substance NMDA, the assay described below was employed.

Hippocampal Slice Cultures

Slice cultures were prepared and grown by the interface method (Stoppini L et al, J Neurosci Methods, 1991; 37(2):173-82). Briefly, seven-day-old Wistar rats (Moellegaard, Denmark) were killed by decapitation and the brain removed under aseptic conditions. After isolation of the hippocampus, transverse sections were cut at 350 μm by a McIlwain tissue chopper, were transferred to Geys balanced salt solution (GIBCO-BRL, Life Technologies, Denmark) for separation and then placed on semiporous Millipore membranes in plastic inserts (Millicell-CM 0.4 μm, 30 mm diameter, Millipore Corporation Bedford, USA). Six hippocampal tissue slices were equally spaced on each insert, which was transferred to 6-well culture trays with 1 ml of growth medium in each well. The medium was composed of 25 ml Hanks BSS, 50 ml OPTI-MEM and 25 ml horse serum (all GIBCO-BRL, Life Technologies, Denmark), supplemented by 1 ml 50% D (+) glucose monohydrate (Merck, Germany). The culture trays were placed at 36° C. in an incubator with 5% CO₂ and 100% humidity in atmospheric air. After 3 days, the culture medium was totally replaced with 1 ml serum-free, chemical defined Neurobasal medium, with addition of 2 ml B27 supplement (both GIBCO-BRL, Life Technologies, Denmark) and 500 μl L-glutamine (Sigma-Aldrich, Denmark) per 98 ml Neurobasal medium. For the next 2-3 weeks, the medium was changed twice a week with regular microscopically inspection of the cultures. Only cultures with intact and well-defined hippocampal neuronal layers were subsequently analyzed.

Supply of NsG-3

For supply of NsG-3, conditioned medium from cultures of transfected NsG-3-producing HiB5-cells was used due to the novelty and the shortage of recombinant human NsG-3. Two clones of NsG-3 transfected HiB5 cells were used: HiB5-NsG3g-2 or HiB5-NsG3c-4, transfected by a genomic or cDNA construct, respectively. As negative control, medium from non-transfected HiB5 cells was used. To obtain the control and NsG-3-containing media, transfected or non-transfected HiB5 cells were grown for 2-4 days in a medium composed of 150 ml D-MEM (Gibco), 16.7 ml heat-inactivated horse serum (Gibco), and 2 ml 0.47 mg/ml hexamycin (Durascan, Odense, Denmark), before samples of the media were used for the experiments (see below).

Exposure to NMDA

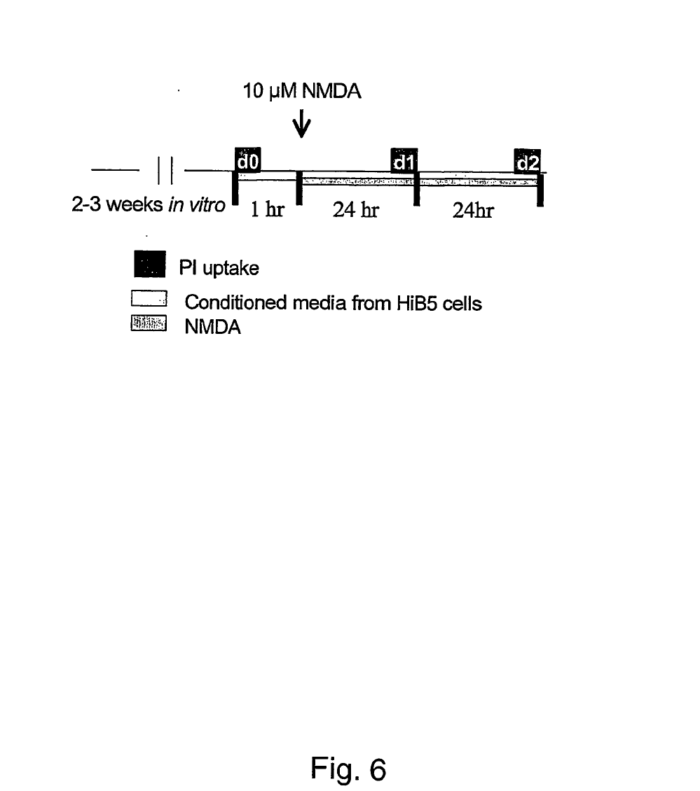

Hippocampal slice cultures were exposed to 10 μM NMDA for 48 hrs to induce a relative selective degeneration of CA1 pyramidal cells (Kristensen B W, Noraberg J. Ebert B et al. Restor Neurol Neurosci 2000; 16: 26-27). One hour before the exposure to NMDA, some cultures had the regular serum-free medium changed to medium taken from cultures of transfected NsG-3-producing or non-transfected HiB5 cells (see above). A separate group of cultures were not exposed to NMDA and had the regular serum-free medium changed to conditioned medium non-transfected HiB5 cells. This group served as a control. The entire experimental protocol is shown schematically in FIG. 6.

Quantitation of Neuronal Death: Propidium Iodide (PI) Uptake

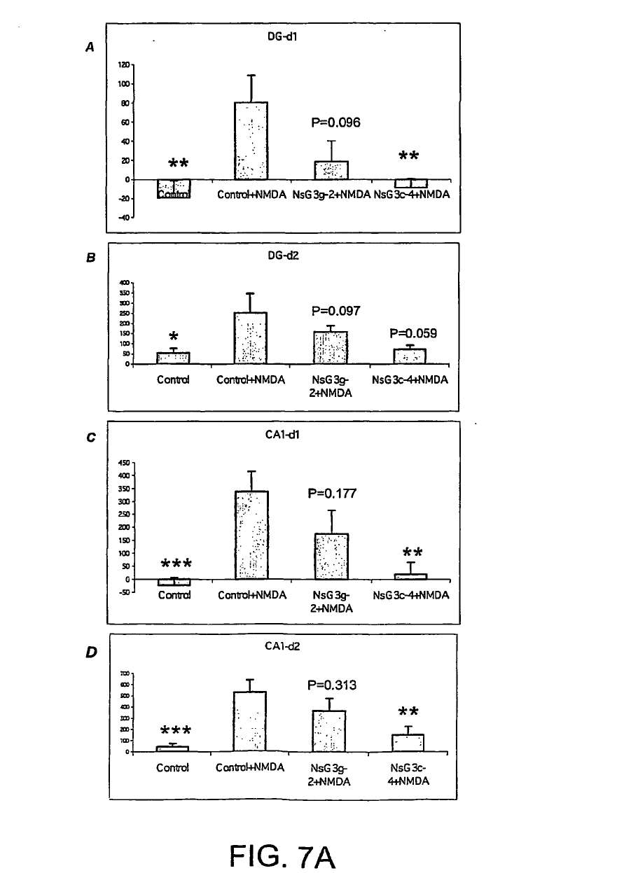

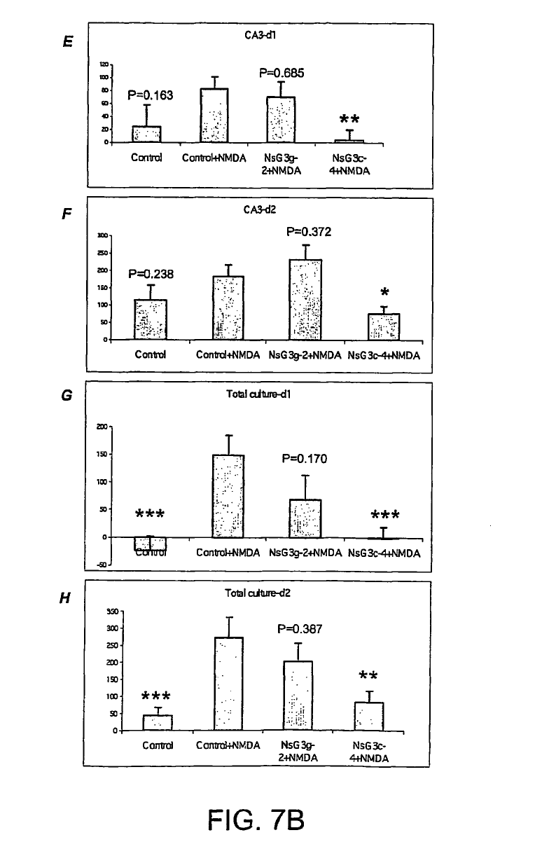

Neuronal degeneration was monitored by densitometric measurements of the cellular uptake of Propidium Iodide (PI; Sigma), which is a polar compound that only enters dead or dying cells with damaged membranes. Once inside the cell, PI binds to nucleic acid with a strong red fluorescence (630 nm) when excited by green light (495 nm). At a concentration of 2 μM, PI is basically non-toxic to neurons (Macklis J D and Madison R D. J Neurosci Methods 1990; 31: 4346). In the present experiments 2- to 3-week-old hippocampal slice cultures were exposed to 2 μM PI, by addition to the medium at least 3 h before exposure to 10 μM NMDA. PI uptake was recorded by a digital camera, Sensys KAF 1400 G2 (Photometrics, Tucson, Ariz., USA) before the addition of the conditioned HiB5 media and then 24 h (day 1/“d1” in FIG. 6) and 48 h (day 2/“d2” in FIG. 6) after start of NMDA exposure. The PI uptake was quantified by densitometric analysis, using NIH Image software version 1.62 (Noraberg J. Kristensen B W and Zimmer J. Brain Res Protocols 1999; 3: 278-290). The densitometric analysis was performed for the dentate gyrus (DG) and the subfields CA1 and CA3 within the tissue slices as well as for the total culture (see TABLE 3 and FIGS. 7A and B).

Table 3 shows the statistical significance of recorded PI uptake levels relative to control culture, which was exposed to NMDA and conditioned medium from non-transfected HiB5 cells. The statistical significance was obtained by one-way ANOVA and Bonferroni post-test. Number of replicates per treatment type n=10-17.

TABLE 3
Excitotoxic_effects

| | Control | NsG3g-2 + | NsG3c-4 + |
|---|---|---|---|
| Neuron type/day-after- | (No NMDA) | NMDA | NMDA |
| NMDA | P-value | P-value | P-value |
| DG/day1 | ** | 0.096 | ** |
| DG/day2 | * | 0.097 | 0.059 |
| CA1/day1 | *** | 0.177 | ** |
| CA1/day2 | *** | 0.313 | ** |
| CA3/day1 | 0.163 | 0.685 | ** |
| CA3/day2 | 0.238 | 0.372 | * |
| Total culture/day1 | *** | 0.170 | *** |
| Total culture/day2 | *** | 0.387 | ** |

P-values are shown as ‘*’ (P < 0.05), ‘**’ (p < 0.01), or ‘***’ (P < 0.001).

FIGS. 7A and B show densitometric measurements of propidium iodide (PI) uptake at time-points day 1 (d1) and day 2 (d2) in dentate gyrus (DG; panels A+B), in the hippocampal subfields CA1 (panels C+D) and CA3 (panels E+F), and in total culture (panels G+H). Control=cultures exposed to control HiB5 conditioned medium without added NMDA; Control+NMDA=cultures pre-exposed 1 hr to control HiB5 conditioned medium followed by 10 μM NMDA; NsG3g-2+NMDA=cultures pre-exposed 1 hr to NsG-3g-2 (genomic fragment) transfected HiB5 cells followed by 10 μM NMDA; NsG3c4+NMDA=cultures pre-exposed 1 hr to NsG-3c4 (cDNA) transfected HiB5 cells followed by 10 μM NMDA. Bars represent the average value of the PI fluorescence recorded on the given day subtracted by the value of PI fluorescence recorded before the start of the experiment (d0)±S.E.M. Values are given in arbitrary units. Statistical significance was obtained by one-way ANOVA and Bonferroni post-test (*P<0.05, **P<0.001, ***P<0.001). Number of replicates per treatment type n=-10-17.

Effects of NsG-3 on NMDA-Induced Excitotoxic Injury

The addition of conditioned medium from HiB5 cells expressing NsG-3 cDNA was found to lower the amount of NMDA-induced cell death to a level comparable to tissue slices which did not receive NMDA at all (Control). The effect was most noticeable in hippocampal subfields CA1 and CA3 and was observed both one and two days after NMDA exposure (TABLE 3 and FIGS. 7A and B). The effects of conditioned medium arising from HiB5 cells expressing a NsG-3 genomic construct were lower than those observed from the cDNA-based NsG-3 construct but still indicated some effect, in particular in the dentate gyrus area. The reason for these observations may be that the cDNA-based construct leads to higher amounts of the correct full-length form of the NsG-3 protein being produced and secreted.

### Example 6

### Receptor Activation by NsG-3

To evaluate the ability of NsG-3 to activate receptor complexes belonging to the TGF-beta receptor subfamilies type I and type II, the following studies were performed.

Luciferase Reporter Assays

For luciferase reporter assays, HepG2 cells were cultured in 24-well plates and transfected with plasmid DNA using Fugene-6 (Roche) (Reissmann E et al. (2001) Genes Dev. 15:2010-22.). To control for cell number and transfection efficiency, Renilla luciferase under a minimal cytomegalovirus promoter (PRL-CMV, Promega) was included in the transfection mix. All transfections were done in triplicate with a total amount of 1 μg of DNA per three wells. Thirty-six hours after transfection, luciferase activity was analyzed using the Dual-Luciferase Reporter Assay System (Promega) in a 1450 Microbeta let counter (Wallac).

Transfection Studies

HepG2 cells were transiently transfected in various combinations with the following plasmid constructs (see more experimental details in Reissmann E et al. (2001) Genes Dev. 15:2010-22.)

pRL-CMV: Control luciferase construct

pCAGA-1uc: Smad3-specific multimerized reporter construct containing a (CAGA)₉ nine-tandem copy in front of the luciferase gene

pNsG-3: Vector containing NsG-3 cDNA (SEQ ID NO:2) under control by a eukaryotic promoter, e.g. the Ubiquitin promoter used in vector pNsG-3-c4 described in Example No. 4)

pALK-4: Vector containing cDNA for the ALK4 type I receptor under control by a eukaryotic promoter

pALK-7: Vector containing cDNA for the ALK7 type I receptor under control by a eukaryotic promoter

pActRIIB: Vector containing cDNA for the ActRIIB type II receptor under control by a eukaryotic promoter

NsG-3 Activates a Combination of Receptor ALK7 or ALK4 and Receptor ActRIIB

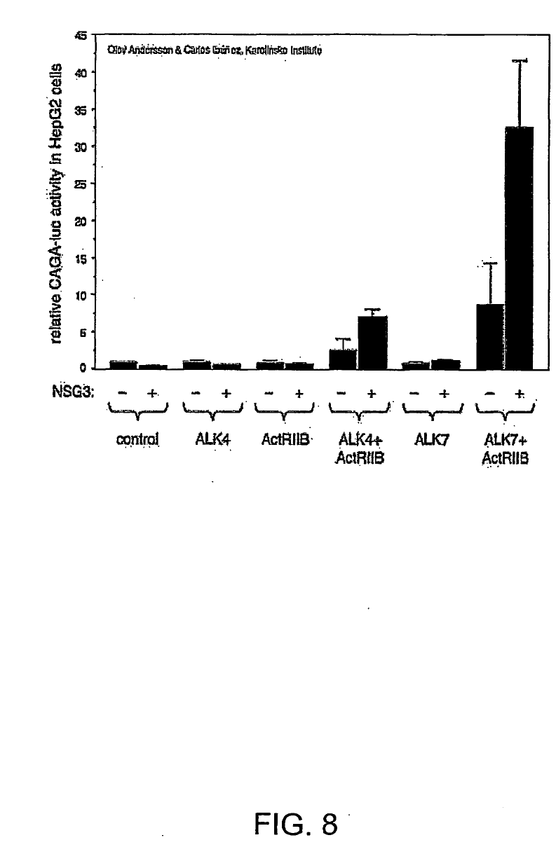

Results from a luciferase assay are shown in FIG. 8. High levels of luciferase activity indicates that the Smad-3 specific signal transduction pathway was activated. Smad-3 binds to the CAGA nine-tandem copy element present in reporter plasmid pCAGA-1uc. A constitutively active form of the ALK7 receptor has been shown to signal via Smad-3 activation (Jörnvall H et al. (2001) J Biol Chem. 276: 5140-6).

Results from different combinations of expressing different receptors with or without NsG-3 is shown in FIG. 8. Expressing NsG-3 alone, ALK4 receptor+/−NsG-3, ActRIIB+/−NsG-3, or ALK7+/−NsG-3 showed no increase in luciferase activity. Expression of either ALK4 or ALK7 in combination with the type II receptor ActRIIB showed some basal activity when NsG-3 was absent. When ActRIIB and NsG-3 was co-expressed, the luciferase activity dramatically increased from approx. 2 to 7 arbitrary units for ALK4 and from approx. 9 to 33 arbitrary units for ALK7.

Experiments using the same receptor combinations but using GDNF instead of NsG-3 as the ligand showed no effect (data not shown). Similar experiments using a combination of NsG-3 and receptors ALK5 and TBRII also showed no effect (data not shown).

The results indicate that NsG-3 acts as a cognate ligand of the type I ALK7 receptor. The ALK7 receptor has been shown to be expressed almost exclusively in the adult central nervous system, in particular the cerebellum and hippocampus (Rydén Met al (1996) J Biol Chem. 271:30603-9). This expression pattern supports the finding that NsG-3 has a s neuroprotective effect on hippocampal neurons as described in Example 4.

1. A polypeptide having the amino acid sequence of any of SEQ ID NO: 5, 9, 11, 13, or 15, a variant of one of the said sequences having neuroprotective activity and having at least 90% identity with any of said SEQ Ids, and having between 90 and 188 amino acids, and said variant comprising the 7 conserved amino acid of the TGF-beta superfamily corresponding to amino acids no. 15, 44, 46, 48, 77, 109 and 111 of SEQ ID NO: 11.

2. A polypeptide according to claim 1, wherein the neuroprotective effect is on NMDA-induced neuronal degradation.

3. A polypeptide according to claim 1 having 97-180amino acids.

4. A polypeptide according to claim 1 having the amino acid sequence of SEQ ID NO:11 or a variant thereof having neuroprotective effect and having at least 90% sequence identity therewith.

5. An isolated polynucleotide encoding a polypeptide having neuroprotective activity, wherein the polynucleotide has 1) a sequence according to any of SEQ ID NO: 4, 8, 10, 12, or 14, 2) a variant of one of the said sequences having at least 30% identity therewith and encoding a polypeptide according to claim 1.

6. A recombinant expression vector construct comprising a polynucleotide according to claim 5.

7. A recombinant host cell comprising the recombinant expression vector construct according to claim 6.

8. A method of producing a polypeptide comprising culturing the host cell according to claim 7 in a suitable culture medium under conditions conducive to expression of the polypeptide, and recovering the polypeptide from the culture medium.

9. A pharmaceutical composition comprising as an active substance a polypeptide having the amino acid sequence of any of SEQ ID NO: 3, 5, 7, 9, 11, 13, or 15, or a variant of one of the said sequences having neuroprotective activity and having at least 50% identity with any of said SEQ Ids and said variant comprising between 90 and 188 amino acids and said variant comprising the 7 conserved amino acid of the TGF-beta superfamily corresponding to amino acids no. 15, 44, 46, 48, 77, 109 and 111 of SEQ ID NO: 11.

10-15 (Canceled)

16. A packaging cell line capable of producing an infective virion comprising a vector construct, said vector construct comprising
1) a polynucleotide according to any of SEQ ID No 2, 4, 6, 8, 10, 12, or 14;
2) a variant of one of the said sequences having at least 30% sequence identity therewith and encoding a neuroprotective polypeptide having the amino acid sequence of any of SEQ ID NO: 3, 5, 7, 9, 11, 13, or 15, or a variant of one of the said sequences having neuroprotective activity and having at least 50% identity with any of said SEQ IDs and said polypeptide variant comprising between 90 and 188 amino acids and said polypeptide variant comprising the 7 conserved amino acid of the TGF-beta superfamily corresponding to amino acids no. 15, 44, 46, 48, 77, 109 and 111 of SEQ ID NO: 11; or 3; or
3) a polynucleotide sequence which hybridises to any of SEQ ID No 2, 4, 6, 8, 10, 12, or 14 under highly stringent conditions.

17. A pharmaceutical composition comprising the packaging cell line of claim 16, an isolated polynucleotide, an expression vector construct comprising said isolated polynucleotide, or a host cell comprising said expression vector construct, said isolated polynucleotide being selected from the group consisting of:
1) a polynucleotide according to any of SEQ ID No 2, 4, 6, 8, 10, 12, or 14;
2) a variant of one of the said sequences having at least 30% sequence identity therewith and encoding a neuroprotective polypeptide having the amino acid sequence of any of SEQ ID NO: 3, 5, 7, 9, 11, 13, or 15, or a variant of one of the said sequences having neuroprotective activity and having at least 50% identity with any of said SEQ IDs and said polypeptide variant comprising between 90 and 188 amino acids and said polypeptide variant comprising the 7 conserved amino acid of the TGF-beta superfamily corresponding to amino acids no. 15, 44, 46, 48, 77, 109 and 111 of SEQ ID NO: 11; or 3; and
3) a polynucleotide sequence which hybridises to any of SEQ ID No 2, 4, 6, 8, 10, 12, or 14 under highly stringent conditions.

18. A method of treating or preventing a neurodegenerative disease in an animal comprising administering to the animal an effective amount of the polypeptide as defined in claim 9, the packaging cell line of claim 16, an isolated polynucleotide, an expression vector construct comprising said isolated polynucleotide, or a host cell comprising said expression vector construct, said isolated polynucleotide being selected from the group consisting of:
1) a polynucleotide according to any of SEQ ID No 2, 4, 6, 8, 10, 12, or 14;
2) a variant of one of the said sequences having at least 30% sequence identity therewith and encoding a neuroprotective polypeptide having the amino acid sequence of any of SEQ ID NO: 3, 5, 7, 9, 11, 13, or 15, or a variant of one of the said sequences having neuroprotective activity and having at least 50% identity with any of said SEQ IDs and said polypeptide variant comprising between 90 and 188 amino acids and said polypeptide variant comprising the 7 conserved amino acid of the TGF-beta superfamily corresponding to amino acids no. 15, 44, 46, 48, 77, 109 and 111 of SEQ ID NO: 11; or 3; and
3) a polynucleotide sequence which hybridises to any of SEQ ID No 2, 4, 6, 8, 10, 12, or 14 under highly stringent conditions.

19. A method according to claim 18, wherein the neurodegenerative disease is one involving lesioned and/or traumatic neurons.

20. A method according to claim 18, wherein the neurodegenerative disease is one involving traumatic lesions of the peripheral nerves, the medulla, and/or the spinal cord, cerebral ischaemic neuronal damage, neuropathy and especially peripheral neuropathy, Alzheimer's disease, Huntington's disease, Parkinson's disease, amyotrophic lateral sclerosis and memory impairment connected to dementia.

21. A method according to claim 18, wherein the neurodegenerative disease is an excitotoxic disease.

22. A method according to claim 21, wherein the excitotoxic disease is selected from the group consisting of ischaemia, epilepsy and trauma due to injury, cardiac arrest and stroke.

23. A method of treating or preventing an excitotoxic disease in an animal comprising administering to the animal an effective amount of
a polypeptide having the amino acid sequence of any of SEQ ID NO: 3 or 5 or a neuroprotective variant of one of the said sequences having at least 50% identity therewith and comprising between 90 and 188 amino acids and comprising the seven conserved amino acids of the TGF-beta superfamily corresponding to amino acid residues 15, 44, 46, 48, 77, 109 and 111 of SEQ ID NO: 11;
a polynucleotide encoding a neuroprotective polypeptide, wherein the polynucleotide has 1) a sequence according to any of SEQ ID NO: 2 or 4, 2) a variant of one of the said sequences having at least 30% identity therewith and encoding a polypeptide comprising between 90 and 188 amino acids and comprising the seven conserved amino acids of the TGF-beta superfamily corresponding to amino acid residues 15, 44, 46, 48, 77, 109 and 111 of the SEQ ID NO: 11, or 3) a polynucleotide, which hybridises to any of SEQ ID NO: 2 or 4 under highly stringent conditions;
a recombinant expression vector construct comprising the said polynucleotide;
a recombinant host cell comprising the said recombinant expression vector construct; or
a packaging cell line capable of producing an infective virion comprising the said vector construct.

24. A polypeptide according to claim 3, consisting of 97-140 amino acids.

25. A polypeptide according to claim 3, consisting of 97-112 amino acids.
